(19) United States
(12) Patent Application Publication
(10) Pub. No.: US 2007/0004935 A1
(43) Pub. Date: Jan. 4, 2007

(54) Efficient method for the manufacture of (E) -Entacapone polymorphic Form A

(75) Inventors: Siddiqui Mohammed Jaweed Mukarram, Aurangabad (IN); Rashid Abdul Rehman Khan, Aurangabad (IN); Ram Prasad Yadav, Aurangabad (IN)

Correspondence Address: IPLM GROUP, P.A., POST OFFICE BOX 18455, MINNEAPOLIS, MN, 55418, US

(73) Assignee: Wockhardt Limited

(21) Appl. No.: 11/474,732

(22) Filed: Jun. 26, 2006

(30) Foreign Application Priority Data
Dec. 24, 2003 (WO) PCT/IB03/06176

(51) Int. Cl. C07C 255/44

(52) U.S. Cl. 558/410

(57) ABSTRACT

The present invention describes an improved method for the preparation of Entacapone, (E)-N,N-diethyl-2-cyano-3-(3,4-dihydroxy-5-nitrophenyl)acrylamide, polymorphic Form A, which is an inhibitor of catechol-O-methyltransferase (COMT) enzyme. The method provides pure (E)-isomer of Entacapone Form A.

### CROSS-REFERENCE TO RELATED APPLICATION

This application claims priority from International Patent Application Serial No. PCT/IB2003/006176 filed Dec. 24, 2003, and published in English on Aug. 4, 2005 as International Publication No. WO 2005/070881 A1, which are incorporated herein by reference.

### FIELD OF THE INVENTION

The present invention relates to a simple and efficient method for the preparation of stable and crystallographically pure polymorphic Form A of Entacapone, (E)-N,N-diethyl-2-cyano-3-(3,4-dihydroxy-5-nitrophenyl)acrylamide) having Formula I, 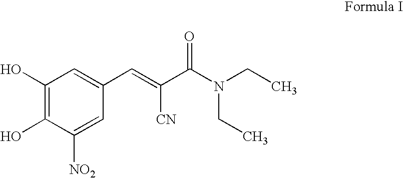
Entacapone is a potent inhibitor of catechol-O-methyl-transferase (COMT) enzyme. The required geometrical (E)-isomer having excellent crystalline purity is obtained during the simple extraction procedure with ethyl acetate solvent once the reaction is complete.

### BACKGROUND OF THE INVENTION

The chemical name for Entacapone is N,N-diethyl-2-cyano-3-(3,4-dihydroxy-5-nitrophenyl)acrylamide. U.S. Pat. No. 4,963,590 and British Patent Application No. 2 200109 A state that Entacapone is a potent inhibitor of catechol-O-methyl-transferase (COMT) enzyme. Entacapone has also been indicated for the treatment of Parkinson's disease. Catechol-O-methyltransferase (COMT) enzyme catalyzes the methyl group from S-adenosyl-L-methionine to a number of compounds with catechol structures. This enzyme is important in the extraneuronal inactivation of catecholamines and drugs with catechol structures. COMT is an important enzyme involved in the metabolism of catecholamines. The COMT enzyme is present in the most tissues, both in periphery and the central nervous system. The highest activities are found in the liver, intestine and kidneys.

U.S. Pat. No. 4,963,590 (“the '590 patent”) discloses a process for the preparation of N,N-diethyl-2-cyano-3-(3,4-dihydroxy-5-nitrophenyl)acrylamide (Formula I). The synthetic process disclosed in the '590 patent includes the condensation of 3,4-dihydroxy-5-nitrobenzaldehyde (Formula II) and N,N-diethylcyano acetamide (Formula III) in anhydrous ethanol as shown below: 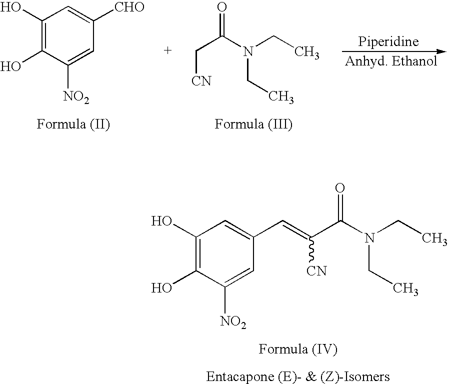
The catalyst used in the process described in the '590 patent synthesis was piperidine acetate. The Entacapone synthesized was obtained in 73% yield having a mixture of two geometrical isomeric forms, i.e., (E)- and (Z)-isomers. The separation of the isomers prepared is not disclosed in the '590 patent.

In addition, U.S. Pat. No. 5,131,950 (“the '950 patent”) discloses that (E)- and (Z)-isomers having the structural formula: 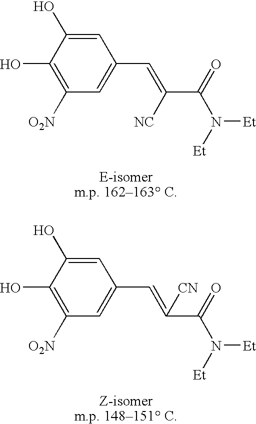
are obtained as mixture in the ratio of about 70-80% to about 30-20%, respectively. As revealed by X-Ray crystallography, (E)-N,N-diethyl-2-cyano-3-(3,4-dihydroxy-5-nitrophenyl)acrylamide may exist at least in two polymorphic forms A and B. The (Z)-isomer, as well as polymorphic form B of the (E)-isomer, have been shown to be unstable under the influence of heat or acids. Similarly, the polymorphic form B of the (E)-isomer isomerizes slowly to the polymorphic form A on standing at room temperature. Crude synthesized Entacapone in conventional solvents, such as hydrocarbons, alcohols or esters, e.g., benzene, toluene, methanol, ethanol, ethyl acetate, isopropyl acetate, etc., provide a very complicated mixture of different geometric isomers and/or polymorphic forms are generally obtained that interfere with the characterization and standardization of the drug substance. The bioavailability of the drug may be also influenced by polymorphism and geometrical isomerism.

The '950 patent discloses that “crystallographically essentially pure” and stable polymorphic form A of (E)-N,N-diethyl-2-cyano-3-(3,4-dihydroxy-5-nitrophenyl)acrylamide may be obtained, when the crude product of synthesis is re-crystallized from lower aliphatic carboxylic acids such as formic acid or acetic acid with a catalytic amount of hydrochloric or hydrobromic acid as shown below: 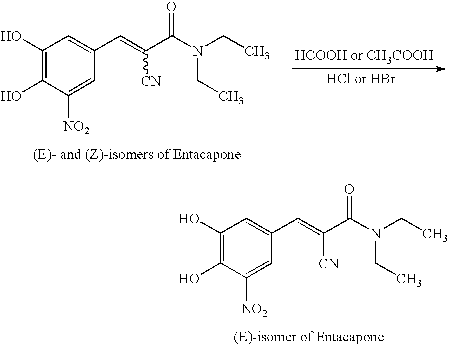
The '950 patent method states that this procedure allows the large scale production of homogeneous and crystallographically essentially pure polymorphic pure form A of (E)-N,N-diethyl-2-cyano-3-(3,4-dihydroxy-5-nitrophenyl)acrylamide. In the '950 patent, “crystallographically essentially pure” is stated to mean that the polymorphic form A of (E)-N,N-diethyl-2-cyano-3-(3,4-dihydroxy-5-nitrophenyl)acrylamide containing a maximum of 3% and preferably a maximum of 2% of other polymorphic forms or the Z-isomer.

The main drawbacks of the '950 patent procedure are:
a) it requires isolation of crude mixture and further treatment of the isomer mixture with formic acid or acetic acid in the presence of hydrochloric acid or hydrobromic acid to obtain isomerically pure (E)-N,N-diethyl-2-cyano-3-(3,4-dihydroxy-5-nitrophenyl)-acrylamide;
b) it uses hazardous and corrosive acids as a solvent for re-crystallization;
c) removal of residual solvent used in the crystallization process may be problematic at large scale operations; and
d) the product obtained can become contaminated with unwanted (Z)-isomer (up to about 2%).

There is a need for an economical and industrially advantageous manufacturing method of (E)-N,N-diethyl-2-cyano-3-(3,4-dihydroxy-5-nitrophenyl)acrylamide substantially free of (Z)-isomer.

### SUMMARY OF THE INVENTION

The present invention provides a simple and efficient method for the manufacture of stable and crystallographically pure polymorphic Form A of (E)-N,N-diethyl-2-cyano-3-(3,4-dihydroxy-5-nitrophenyl)acrylamide (Entacapone) having Formula I, 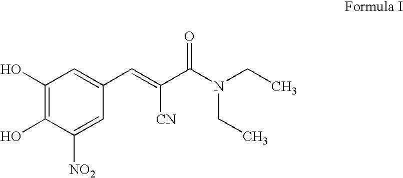
which is a potent inhibitor of catechol-O-methyl-transferase (COMT) enzyme.

In one embodiment, a method is provided for the manufacture of (E)-N,N-diethyl-2-cyano-3-(3,4-dihydroxy-5-nitrophenyl)acrylamide. The compound, 3,4-dihydroxy-5-nitrobenzaldehyde, is condensed with N,N-diethylcyanoacetamide in presence of a base in alcoholic solution.

The geometrical (E)-isomer with excellent polymorphic crystalline purity is obtained after simple extraction method. The crude (E)-Entacapone having both geometrical isomers, (E)- and (Z)- is extracted by ethyl acetate in acidic medium preferably pH between about 3.5 to about 4.0. The isolated product yield of the (E)-isomer is more than 99.6% pure by HPLC.

### DETAILED DESCRIPTION OF THE INVENTION

The present invention provides a method to prepare the stable polymorphic form A of (E)-N,N-diethyl-2-cyano-3-(3,4-dihydroxy-5-nitrophenyl)acrylamide obtained in high purity without isolating crude solid isomeric mixture of N,N-diethyl-2-cyano-3-(3,4-dihydroxy-5-nitrophenyl)acrylamide. The synthetic method for preparing the crude Entacapone is shown below:

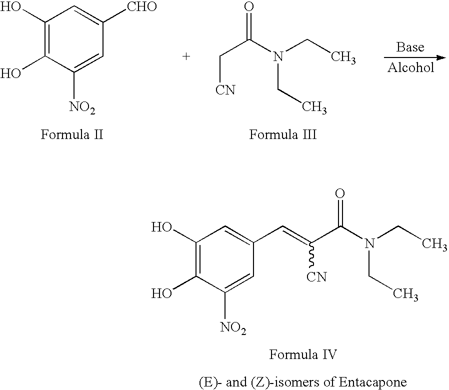

The synthesis is carried out by condensing 3,4-dihydroxy-5-nitrobenzaldehyde (Formula II) with N,N-diethyl cyanoacetamide (Formula III) in alcohol such as methanol, ethanol, isopropanol, isobutanol, n-butanol etc., preferably isopropanol at reflux temperature in the presence of a suitable organic base, such as piperidine, N-methylmorpholine, pyridine, piperazine, etc. Preferably, the reaction is carried out using piperidine as the base. After completion of the reaction, the reaction mixture is poured into a solvent mixture of chilled water and ethyl acetate. The pH of the solution is adjusted to from 3.5 to 4.0 by the addition of an acid. The preferable acid used for acidification is acetic acid. The ethyl acetate layer is separated, washed with water and concentrated to obtain pure (E)-isomer of polymorphic form A, essentially without (Z)-isomer contamination.

In another embodiment, the solvent mixture is substantially free of inorganic halo acids. The inorganic halo acids are hydrofluoric acid, hydrochloric acid, hydrobromic acid or hydroiodic acid.

In another embodiment, the Entacapone is not isolated in its crude form. It is extracted with ethyl acetate which, after concentration, provides the desired product with excellent purity. Other aliphatic esters such as methyl acetate, propyl acetate, or n-butyl acetate provide a similar result.

U.S. Pat. No. 5,135,950 states that the crude mixture can not afford desired purity using lower aliphatic alcohols, esters or hydrocarbons, e.g. ethanol, 2-propanol, ethyl acetate, benzene or toluene and a very complicated mixture of different geometrical isomers and/or polymorphic forms are generally obtained. Surprisingly, the present method provides pure product by very simple operation. HPLC purity of the product of present invention, i.e., (E)-Entacapone is more than 99% while (Z)-isomer is found in less than 0.1%.

The specification of HPLC profile of the present invention is mentioned below:

| | |
|---|---|
| Column: | HICHROM HIRPB (250 × 4.6 m, 5μ) |
| Wavelength: | 210 nm |
| Flow rate: | 1.0 ml/minute |
| Temperature: | Ambient |
| Buffer: | 1 ml H₃PO₄ in 1000 ml distilled water |
| Mobile phase: | Buffer (65): Acetonitrile (35) |
| Sample preparation: | 0.5 mg/ml in mobile phase |
| Retention Time: | (E)-Isomer = 11.5 minutes, |
| | Z-Isomer = 10.3 minutes |

The HPLC method of analysis clearly resolves both the reactants as well as (Z)- and (E)-isomers of Entacapone.

The Infra Red spectrum of polymorphic form A of (E)-N,N-diethyl-2-cyano-3-(3,4-dihydroxy-5-nitrophenyl)acrylamide is characterized by the following peaks at about 3330, 3083, 3059, 3032, 2979, 2935, 2216, 1628, 1606, 1580, 1541, 1512, 1440, 1379, 1296, 1280, 1209, 1165, 1150, 800, 777, 742 and cm⁻¹.

The X-Ray powder diffraction of the Entacapone prepared by the method of the present invention is characterized by the following peaks (2θ) at about 9.080, 11.700, 11.880, 13.600, 14.140, 15.860, 16.400, 18.240, 18.600, 18.800, 19.040, 20.080, 20.460, 21.200, 21.980, 22.600, 23.040, 23.440, 23.960, 26.160, 26.920, 27.500, 28.000, 28.580, 29.140, 30.200, 30.740, 32.060, 32.580 and 33.320°.

The merits of the present invention over other processes are as follows:
a) a single step process provides highly pure (E)-N,N-diethyl-2-cyano-3-(3,4-dihydroxy-5-nitrophenyl)acrylamide without isolation of crude mixtures of isomers and re-crystallizations process;
b) purity is ascertained by HPLC analysis and getting unknown impurity in less than 0.1%; and
c) the process is robust and adoptable at kilogram to multi kilogram level.

The example discussed below illustrates the invention, but is not limiting thereof.

### EXAMPLE 1

### (E)-N,N-Diethyl-2-cyano-3-(3,4-dihydroxy-5-nitrophenyl)acrylamide

3,4-Dihydroxy-5-nitrobenzaldehyde (5.0 gm, 27.3 mmol), N,N-diethyl cyanoacetamide (7.65 gm, 54.57 mmol) and piperidine (7.50 gm, 85.15 mmol) are charged to isopropanol (50 ml). The reaction mixture is heated at reflux for 10 to 15 hours till the disappearance of the starting material. After the reaction is complete, the reaction mixture is cooled to room temperature. The cooled reaction mixture is poured slowly into a mixture of cold water and ethyl acetate, followed by adjusting the pH to about 3.5 to about 4.0 with acetic acid. The organic layer is separated, filtered through activated charcoal and concentrated to provide crystalline (E)-N,N-diethyl-2-cyano-3-(3,4-dihydroxy-5-nitrophenyl)acrylamide. The title product is filtered and dried to obtain the desired product in 99.7% HPLC purity. Mass spectra=(m+1) 306 (100%). (Z)-Entacapone is observed in less than 0.1%.

All references cited herein are expressly incorporated herein by reference in their entirety into this disclosure. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure and the claims shown below are not limited to the illustrative embodiments set forth herein.

1. A method for preparing a compound of Formula (I): 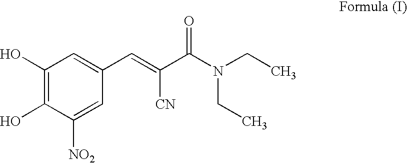
from a mixture of the (E)- and (Z)-isomers of Formula (I) comprising:
(a) contacting the mixture of isomers with an aliphatic acid in a solvent mixture; and
(b) isolating the (E)-isomer.

2. The method of claim 1, wherein the solvent mixture is substantially free of inorganic halo acids.

3. The method of claim 2, wherein the inorganic halo acids are hydrofluoric acid, hydrochloric acid, hydrobromic acid, or hydroiodic acid.

4. The method of claim 1, wherein the solvent mixture comprises water and an organic ester.

5. The method of claim 4, wherein the organic ester is methyl acetate, ethyl acetate, n-propyl acetate or n-butyl acetate.

6. The method of claim 5, wherein the ester is ethyl acetate.

7. The method of claim 1, wherein the aliphatic acid is acetic acid.

8. The method of claim 1, wherein the solvent layer is filtered through activated charcoal.

9. The method of claim 1, wherein the N,N-diethyl-2-cyano-3-(3,4-dihydroxy-5-nitrophenyl)acrylamide is crystallized from the solvent mixture.

10. The method of claim 1, wherein isolated (E)-N,N-diethyl-2-cyano-3-(3,4-dihydroxy-5-nitrophenyl)acrylamide in step (b) has more than 99.6% HPLC purity.

11. The method of claim 1, wherein the amount of (Z)-N,N-diethyl-2-cyano-3-(3,4-dihydroxy-5-nitrophenyl)acrylamide remaining is less than 0.1%.

12. The method of claim 1, wherein the (E)-N,N-diethyl-2-cyano-3-(3,4-dihydroxy-5-nitrophenyl)acrylamide is polymorph A.

13. The method of claim 3, wherein the inorganic halo acids are hydrochloric acid, or hydrobromic acid.
